(19) United States
(12) Patent Application Publication
(10) Pub. No.: US 2025/0191717 A1
(43) Pub. Date: Jun. 12, 2025

(54) METHOD AND SYSTEM FOR PROVIDING A CONTRAST AGENT ALERT

(71) Applicant: Siemens Healthineers AG, Forchheim (DE)

(72) Inventors: Bijesh BALACHANDRAN, Bangalore (IN); Akshay GANESHA, Bengaluru (IN); Bijaya Kumar MEHER, Sonepur (IN); Supriya SEEMANAPALLI, Bangalore (IN)

(73) Assignee: Siemens Healthineers AG, Forchheim (DE)

(21) Appl. No.: 18/973,173

(22) Filed: Dec. 9, 2024

(30) Foreign Application Priority Data
Dec. 11, 2023 (IN) 202311084495
Feb. 20, 2024 (DE) 10 2024 201 526.1

(51) Int. Cl. G16H 20/10; A61B 6/00; G06T 7/00; G06T 7/62; G06V 30/10; G06V 30/42; G16H 30/40

(52) U.S. Cl. CPC G16H 20/10; G06T 7/0014; G06T 7/62; G06V 30/10; G06V 30/42; G16H 30/40; A61B 6/481; G06V 2201/03; G06V 2201/10

(57) ABSTRACT

One or more example embodiments relates to providing a contrast agent alert in a medical imaging procedure. Medical imaging data related to a medical imaging procedure is received, wherein a contrast agent was administered to a patient during the medical imaging procedure. A volume of contrast agent administered to the patient is determined based on the medical imaging data. A reference volume of contrast agent is determined based on the medical imaging data. An alert is provided if the volume of contrast agent administered fulfills a condition, wherein the condition is based on the reference volume of contrast agent.

### CROSS-REFERENCE TO RELATED APPLICATION(S)

The present application claims priority under 35 U.S.C. § 119 to Indian Patent Application No. 202311084495, filed Dec. 11, 2023, and German Patent Application No. 10 2024 201 526.1, filed Feb. 20, 2024, the entire contents of each of which are incorporated herein by reference.

### FIELD

This patent application pertains to a comprehensive contrast management solution designed to significantly enhance patient care in radiology departments. The invention addresses a technical problem in the field of medical imaging, specifically in procedures involving computed tomography (CT) and magnetic resonance imaging (MRI) that utilize contrast enhancement.

### RELATED ART

Contrast enhancement is typically administered using automated injection systems, commonly referred to as “power injectors”. These systems are routinely used for iodinated contrast agents in interventional settings. While contrast agents are generally safe, adverse events can occur in patients with renal issues. Furthermore, mistimed scans or technical problems during injection can lead to suboptimal images, particularly with fast CT scanners.

The current methods of managing contrast injection data involve manual or semi-automatic procedures, which are often error-prone and lack consistency in documentation. These methods include manual input of information such as contrast volume, flow rates, and injection timing into the CT scanner's system by radiology technologists or healthcare professionals. Electronic health records (EHR) or radiology information systems (RIS) are often employed for data entry during or after the procedure.

### SUMMARY

One or more example embodiments automates the collection of contrast injection data, thereby enhancing compliance, safety, and quality assurance in imaging facilities. This is achieved by establishing an industry-standard information object for consistent access to per-study contrast media data. The solution provides actionable insights for continuous evaluation of contrast usage for different types of injectors or suppliers, and for all protocols used. It also features in-built interoperability capabilities based on communication standards (DICOM and HL7), enabling it to interface with systems like injectors and hospital information systems (PACS, RIS, and EMR). Additionally, the solution offers benchmarking capabilities with data coming in from multiple tenants.

In summary, one or more example embodiments provides a comprehensive contrast management solution that automates the collection of contrast media injection data, thereby enhancing the quality of patient care, improving diagnostic yield of contrast-enhanced imaging procedures, and ensuring compliance with documentation, safety, and quality mandates in radiology departments.

### BRIEF DESCRIPTION OF THE DRAWINGS

The properties, features and advantages of this invention described above, as well as the manner they are achieved, become clearer and more understandable in the light of the following description and embodiments, which will be de-scribed in detail in the context of the drawings. This following description does not limit the invention on the contained embodiments. Same components or parts can be labeled with the same reference signs in different figures. In general, the figures are not for scale.

The numbering and/or order of method steps is intended to facilitate understanding and should not be construed, un-less explicitly stated otherwise, or implicitly clear, to mean that the designated steps have to be performed according to the numbering of their reference signs and/or their order within the figures. In particular, several or even all of the method steps may be performed simultaneously, in an overlapping way or sequentially.

In the following:

FIG. 1 displays an embodiment of a system for providing a contrast agent alert as integrated into the hospital infrastructure,

FIG. 2 displays a first visualization of the contrast agent admitted within one medical imaging procedure,

FIG. 3 displays a second visualization of the contrast agent admitted within one medical imaging procedure,

FIG. 4 displays a third visualization of the contrast agent admitted within one medical imaging procedure,

FIG. 5 displays a flowchart of an embodiment of a method for providing a contrast agent alert, and

FIG. 6 displays a system for providing a contrast agent alert according to an embodiment of the invention.

### DETAILED DESCRIPTION

In the following, solutions are described with respect to the claimed systems as well as with respect to the claimed methods. Features, advantages or alternative embodiments herein can be assigned to the other corresponding claimed objects and vice versa. In other words, the systems can be improved with features described or claimed in the context of the corresponding method. In this case, the functional features of the methods are embodied by objective units of the systems.

In the following, the term “in particular” is used to indicate an optional and/or advantageous additional feature. Furthermore, the terms “applying a neural network to data” or “applying a unit to data” is used to indicate that the respective data is used as input data for the model or the unit, or that input data is used that comprises the respective data (and potentially other data).

One or more example embodiments relates to a computer-implemented method for providing a contrast agent alert. The method comprises receiving medical imaging data related to a medical imaging procedure, wherein during the medical imaging procedure contrast agent was administered to a patient. The method furthermore comprises determining a volume of contrast agent administered to the patient based on the medical imaging data. The method furthermore comprises determining an reference volume of contrast agent based on the medical imaging data. The method furthermore comprises providing an alert if the volume of contrast agent administered fulfills a condition, wherein the condition is based on the reference volume of contrast agent.

Medical imaging data, as used herein, may refer to any data or information obtained from a medical imaging procedure. This could include, but is not limited to, raw data, processed data, metadata, or any combination thereof. The data may be derived from various types of medical imaging modalities such as X-ray, ultrasound, computed tomography (CT), magnetic resonance imaging (MRI), positron emission tomography (PET), or any other imaging modality known in the art. The data may be in various formats, such as DICOM, JPEG, PNG, or any other suitable format. The data may also include patient-specific information, such as patient demographics, medical history, or any other relevant information.

A medical imaging procedure, as used herein, may refer to any process or method used to obtain medical imaging data. This could include, but is not limited to, the use of various imaging modalities such as X-ray, ultrasound, CT, MRI, PET, or any other imaging modality known in the art. The procedure may involve the use of a contrast agent to enhance the visibility of certain structures or fluids within the body. The procedure may also involve the acquisition of a reference volume of contrast agent for comparison purposes. The procedure may be performed in various settings, such as a hospital, clinic, or any other suitable location.

A contrast agent, as used herein, may refer to any substance used to enhance the visibility of structures or fluids within the body during a medical imaging procedure. This could include, but is not limited to, iodine-based agents, barium-based agents, gadolinium-based agents, or any other suitable agents known in the art. The contrast agent may be administered in various ways, such as orally, intravenously, or any other suitable method. The contrast agent may also be used in various amounts, which may be determined based on a reference volume of contrast agent.

A reference volume of contrast agent, as used herein, may refer to a predetermined amount of contrast agent used as a standard or benchmark during a medical imaging procedure. This could include, but is not limited to, a specific volume, weight, concentration, or any other suitable measure of the contrast agent. The reference volume may be determined based on various factors, such as the type of contrast agent, the imaging modality, the patient's weight, or any other relevant factor. The reference volume may be used to ensure the optimal visibility of structures or fluids within the body.

An alert, as used herein, may refer to any notification or warning generated in response to a specific event or condition during a medical imaging procedure. This could include, but is not limited to, an alert generated when the volume of contrast agent used exceeds the reference volume, an alert generated when the medical imaging data indicates a potential medical issue, or any other suitable alert. The alert may be presented in various ways, such as a visual alert, an auditory alert, a tactile alert, or any other suitable method. The alert may be designed to prompt a response from a user, such as a healthcare professional.

A condition, as used herein, may refer to a function that results in a binary variable, in several categorial variable or in a continuous variable. In particular, a condition can relate to the fact whether a variable is higher or lower than a certain threshold.

By monitoring the volume of contrast agent administered and comparing it with a reference volume, the method can provide an alert if the administered volume fulfills a certain condition. This can prevent potential harm to the patient due to overexposure to the contrast agent, thus enhancing patient safety. This can also increase the efficiency of medical imaging procedures, and can save time for healthcare professionals and allow for more patients to be seen.

According one or more example embodiments, the medical imaging data comprises a secondary capture image, wherein determining an volume of contrast agent administered comprises the steps of performing an optical character recognition within the secondary capture image, and determining the volume of contrast agent administered based on recognized characters within the secondary capture image.

A secondary capture image, as used herein, may refer to any image that is converted from a non-DICOM (Digital Imaging and Communications in Medicine) format to a modality-independent DICOM format. This could include, but is not limited to, images captured from various types of equipment such as video interfaces that convert an analog video signal into a digital image, medical imaging devices, or any other suitable equipment known in the art. The secondary capture image may be a grayscale image, a color image, a multi-frame image, or any other suitable type of image. The secondary capture image may be used in various applications, such as medical imaging, telemedicine, or any other suitable application.

Optical character recognition (OCR), as used herein, may refer to any process or method used to convert different types of documents, such as scanned paper documents, PDF files or images captured by a digital camera, into editable and searchable data. This could include, but is not limited to, the use of various OCR technologies such as pattern recognition, feature recognition, or any other suitable OCR technology known in the art. The OCR process may involve various steps, such as pre-processing, character segmentation, character recognition, and post-processing. The OCR process may be used in various applications, such as document digitization, data entry automation, or any other suitable application. The OCR process may be performed on various types of data, such as text data, image data, or any other suitable type of data.

By utilizing optical character recognition (OCR) within the secondary capture image to determine the volume of contrast agent administered, the system can accurately identify and interpret the relevant data from the image. This can lead to a more precise determination of the contrast agent volume, thereby improving the accuracy of the alert system and potentially enhancing patient safety.

According to one or more example embodiments, the method furthermore comprises determining a planned volume of contrast agent based on recognized characters within the secondary capture image, wherein the condition and/or the reference volume of contrast agent is based on the planned volume of contrast agent.

A planned volume of contrast agent, as used herein, may refer to a predetermined amount of contrast agent that is intended to be used during a medical imaging procedure. This could include, but is not limited to, a specific volume, weight, concentration, or any other suitable measure of the contrast agent. The planned volume may be determined based on various factors, such as the type of contrast agent, the imaging modality, the patient's weight, the patient's medical condition, or any other relevant factor. The planned volume may be used to ensure the optimal visibility of structures or fluids within the body during the imaging procedure. The planned volume may be adjusted as necessary, based on the actual conditions encountered during the procedure. The planned volume of contrast agent may also be compared to a reference volume of contrast agent or an actual volume of contrast agent used during the procedure, and an alert may be generated if the planned volume and the reference or actual volume differ by more than a predetermined amount.

By using the planned volume of contrast agent, the knowledge about the patient that led to the planned volume can be incorporated into the alerting procedure.

According to one or more example embodiments, the medical imaging data comprises a structured report document, wherein the structured report document comprises a data element related to the volume of contrast agent administered.

A structured report document, as used herein, may refer to a digital or physical document that organizes data or information in a standardized, predictable manner. This could include, but is not limited to, medical reports, research reports, technical reports, or any other suitable type of report. The structured report document may contain various types of data or information, such as text, images, tables, graphs, or any other suitable type of data or information. The structured report document may be generated using various types of software or systems, such as a reporting system, a document management system, or any other suitable software or system. The structured report document may be stored in various formats, such as DICOM SR (Digital Imaging and Communications in Medicine Structured Reporting), HL7 CDA (Health Level Seven Clinical Document Architecture), PDF, or any other suitable format. The structured report document may be used in various applications, such as medical imaging, clinical trials, patient care, or any other suitable application. The structured report document may also include various types of metadata, such as the date and time of creation, the author, the patient's information, or any other suitable metadata.

DICOM SR, or Digital Imaging and Communications in Medicine Structured Reporting, as used herein, may refer to a standard for the communication and management of medical imaging information and related data. This could include, but is not limited to, the representation, storage, retrieval, and exchange of medical images, structured reports, and other related information. DICOM SR provides a standardized structure for reporting and communicating information derived from medical images, such as measurements, annotations, and other types of data. The structured reports may be associated with various types of medical imaging data, such as data obtained from X-ray, ultrasound, computed tomography (CT), magnetic resonance imaging (MRI), positron emission tomography (PET), or any other imaging modality known in the art. The structured reports may be used in various applications, such as medical imaging, patient care, clinical trials, or any other suitable application. The structured reports may also include various types of metadata, such as the date and time of creation, the author, the patient's information, or any other suitable metadata.

By using a structure report document, information about the planned or administered contrast agent volume is directly accessible, and the determinations of the volume can be done with less errors.

According to one or more example embodiments, the structured report document comprises a data element related to a planned volume of contrast agent, wherein at least one of the condition and the reference volume of contrast agent is based on the planned volume of contrast agent.

According to one or more example embodiments, the structured report document furthermore comprise a data element related to at least one of injection details, syringe pump details, flow rate details, and/or pressure data details, wherein at least one of the volume of contrast agent administered and the reference volume of contrast agent is determined based on said data element.

Injection details, as used herein, may refer to any information related to the administration of a contrast agent during a medical imaging procedure. This could include, but is not limited to, the type of contrast agent used, the volume of contrast agent administered, the method of administration (e.g., intravenous, oral), the location of administration, the timing of administration, or any other relevant information. The injection details may also include information about the use of a syringe pump, such as the model of the pump, the settings used, or any other relevant information.

Syringe pump details, as used herein, may refer to any information related to the use of a syringe pump during the administration of a contrast agent in a medical imaging procedure. This could include, but is not limited to, the model of the pump, the settings used (e.g., flow rate, pressure), the type of syringe used, the volume of contrast agent in the syringe, or any other relevant information.

Flow rate details, as used herein, may refer to any information related to the rate at which a contrast agent is administered during a medical imaging procedure. This could include, but is not limited to, the initial flow rate, the average flow rate, the maximum flow rate, the time at which the flow rate changes, or any other relevant information. The flow rate details may be determined based on various factors, such as the type of contrast agent, the imaging modality, the patient's weight, or any other relevant factor.

Pressure data details, as used herein, may refer to any information related to the pressure at which a contrast agent is administered during a medical imaging procedure. This could include, but is not limited to, the initial pressure, the average pressure, the maximum pressure, the time at which the pressure changes, or any other relevant information. The pressure data details may be determined based on various factors, such as the type of contrast agent, the imaging modality, the patient's weight, or any other relevant factor.

According to one or more example embodiments, the medical imaging data comprises an axial image. According to this aspect, the method furthermore comprises determining at least one of the volume of contrast agent administered and the reference volume of contrast agent based on the axial image.

An axial image, as used herein, may refer to a type of cross-sectional image that is taken in a horizontal plane through the body during a medical imaging procedure. This could include, but is not limited to, images obtained from various types of medical imaging modalities such as computed tomography (CT), magnetic resonance imaging (MRI), positron emission tomography (PET), or any other imaging modality known in the art. The axial image may be used to visualize various structures or fluids within the body, possibly enhanced by the use of a contrast agent. The axial image may be obtained at various levels of the body, such as the head, chest, abdomen, or any other suitable level. The axial image may be used in various applications, such as diagnosis, treatment planning, monitoring of disease progression, or any other suitable application. The axial image may also be combined with other types of images, such as sagittal or coronal images, to provide a more comprehensive view of the body.

According to one or more example embodiments, the medical imaging data comprises at least two axial images, and the method furthermore comprises selecting one of the at least two axial images based on a contrast bolus time, determining at least one of the volume of contrast agent administered and the reference volume of contrast agent based on the selected one of the at least two axial images.

According to one or more example embodiments, determining a reference volume of contrast agent comprises determining imaging metadata based on the based on the medical imaging data, and querying a database comprising a plurality of reference volumes of contrast agent based on the imaging metadata.

Imaging metadata, as used herein, may refer to any data or information that provides context or additional details about a medical image. This could include, but is not limited to, the date and time the image was taken, the type of imaging modality used (e.g., X-ray, ultrasound, CT, MRI, PET), the settings used during the imaging procedure, the patient's information, the use of a contrast agent, or any other relevant information. The imaging metadata may be stored in various formats, such as DICOM, XML, JSON, or any other suitable format. The imaging metadata may be used in various applications, such as image interpretation, patient care, clinical trials, research, or any other suitable application. The imaging metadata may also be used to search for, sort, or filter images in a database.

A database, as used herein, may refer to any system or software used to store, manage, and retrieve data or information. This could include, but is not limited to, relational databases, object-oriented databases, hierarchical databases, network databases, or any other suitable type of database. The database may contain various types of data or information, such as medical images, imaging metadata, patient records, or any other suitable data or information. The database may be used in various applications, such as medical imaging, patient care, clinical trials, research, or any other suitable application. The database may also include various features, such as search capabilities, sorting capabilities, filtering capabilities, or any other suitable features. The database may be located on a local server, a remote server, a cloud-based server, or any other suitable location.

According to one or more example embodiments, the imaging metadata corresponds to a procedure description of the medical imaging procedure.

A procedure description of the medical imaging procedure, as used herein, may refer to any detailed explanation or outline of the steps, methods, or processes involved in a medical imaging procedure. This could include, but is not limited to, the type of imaging modality used (e.g., X-ray, ultrasound, CT, MRI, PET), the preparation of the patient, the positioning of the patient, the settings used during the imaging procedure, the use and administration of a contrast agent, the acquisition of images, the interpretation of images, or any other relevant information. The procedure description may also include safety precautions, potential risks, expected outcomes, or any other relevant details. The procedure description may be used by healthcare professionals to guide the execution of the imaging procedure, to train new staff, for quality control purposes, or for any other suitable application. The procedure description may be stored in various formats, such as text, video, audio, or any other suitable format, and may be included in a database, a patient record, a structured report document, or any other suitable location.

According to one or more example embodiments, the method comprises determining the body region being subject of the medical imaging procedure, wherein the condition is furthermore based on the body region.

According to one or more example embodiments, the condition is based on at least one of the accumulated or maximal volume of contrast agent administered.

According to one or more example embodiments, the method furthermore comprises anonymizing or pseudonymizing the medical imaging data.

Anonymizing medical imaging data, as used herein, may refer to any process or method used to remove or obscure any identifying information from medical imaging data to prevent the identification of the individual to whom the data pertains. This could include, but is not limited to, the removal or obscuring of personal identifiers such as name, date of birth, social security number, or any other relevant information. The anonymization process may be performed using various techniques, such as data masking, data scrambling, data encryption, or any other suitable technique. The anonymized medical imaging data may be used in various applications, such as research, clinical trials, teaching, or any other suitable application where the identification of the individual is not necessary or desired.

Pseudonymizing medical imaging data, as used herein, may refer to any process or method used to replace identifying information in medical imaging data with a pseudonym or code to prevent the direct identification of the individual to whom the data pertains. This could include, but is not limited to, the replacement of personal identifiers such as name, date of birth, social security number, with a unique identifier or code. The pseudonymization process may be performed using various techniques, such as data masking, data encryption, or any other suitable technique. The pseudonymized medical imaging data may be used in various applications, such as research, clinical trials, patient care, or any other suitable application where the direct identification of the individual is not necessary or desired, but where the ability to link the data back to the individual may be needed under certain conditions.

One or more example embodiments furthermore relates to a system for providing a contrast agent alert, comprising at least one processor configured for:
receiving medical imaging data related to a medical imaging procedure, wherein during the medical imaging procedure contrast agent was administered to a patient,
determining a volume of contrast agent administered to the patient based on the medical imaging data,
determining an reference volume of contrast agent based on the medical imaging data,
providing an alert if the volume of contrast agent administered fulfills a condition, wherein the condition is based on the reference volume of contrast agent.

In particular, the system for providing the contrast agent alert can be configured to execute the method according to one or more example embodiments and their aspects.

One or more example embodiments furthermore relates to a computer program comprising instructions which, when the program is executed by a computer, cause the computer to carry out the method of one or more example embodiments.

One or more example embodiments furthermore relates to a computer-readable storage medium comprising instructions which, when executed by a computer, cause the computer to carry out the method of one or more example embodiments.

The realization of one or more example embodiments by a computer program product and/or a computer-readable medium has the advantage that already existing systems can be easily adapted by software updates in order to work as proposed by one or more example embodiments.

The said computer program products can be, for example, a computer program or comprise another element apart from the computer program. This other element can be hardware, for example a memory device, on which the computer program is stored, a hardware key for using the computer program and the like, and/or software, for example a documentation or a software key for using the computer program.

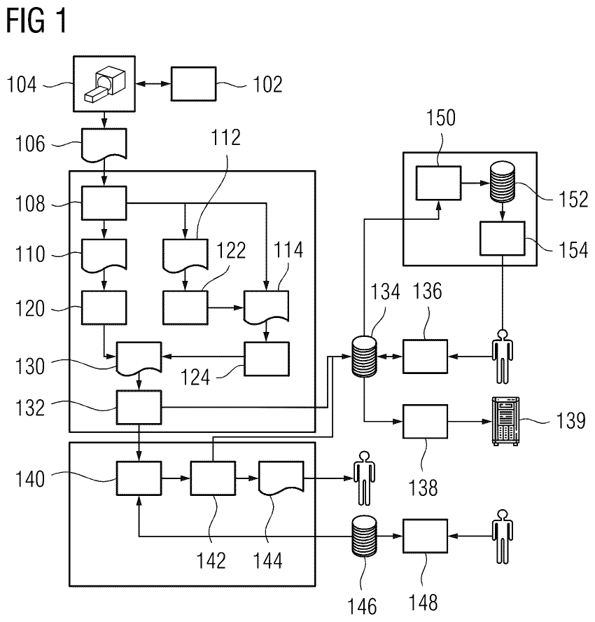

FIG. 1 displays an embodiment of a system for providing a contrast agent alert as integrated into the hospital infrastructure.

In this embodiment, a medical imaging procedure is performed using a medical imaging apparatus 104 and a contrast agent injector 102. Contrast agent injectors 102 can be used independent from their vendor or modality types given the injector is either connected to the medical imaging apparatus 104 and/or the PACS. In this embodiment, the contrast agent injector 102 and the medical imaging apparatus 104 are communicationally coupled. In particular, the contrast agent injector 102 can provide information about the contrast agent injection related to the medical imaging procedure to the medical imaging apparatus 104.

The medical imaging apparatus 104 generates, as a result of the medical imaging procedure, a DICOM study object 106. In particular, a DICOM study object 106 is a collection of all the data, including patient information, descriptions relevant to the examination, and actual image data (such as X-ray, CT, ultrasound), produced during a medical imaging procedure where image data is acquired, and it contains one or more logically related series of medical images, presentation states, and/or structured reports (acronym “SR”) documents for the purpose of diagnosing a patient.

Within the system for providing a contrast agent alert, the DICOM study object 106 is divided into subparts by a data segregation module 108. In particular, the data segregation module 108 extracts from the DICOM study object 106 secondary capture images 112 (black image) of the exam data, contrast structured report documents 114 (acronym “SR”) of the exam data (including but not limited to performed imaging agent administration as per DICOM Supplement 164 and/or planned imaging agent administration as per DICOM Supplement 164) and/or an axial image 110 of the exam data.

If there is an axial image 110 present, it will be further analyzed with an axial image parser 120. In particular, the axial image parser 120 picks the latest axial image by checking the contrast bolus start time if multiple axial images are present, extracts the contrast related information from Axial image and writes all the information to JSON files.

If there is a secondary capture images 112 present, it will be further analyzed with an OCR (acronym for “optical character recognition”) module 122. In particular, the OCR module 122 extracts the contrast information text from the image by OCR, generate structured report documents 114 (planned and performed) from the secondary capture, reads the contrast information from structured report documents 114 and then writes all the information to JSON files.

If there are structure report documents 114 available (either directly within the DICOM study object 106 and/or parsed from a secondary capture image 112, they will be further analyzed with a SR parser module 124. In particular, the SR parser module 124 reads both planned and performed SR and extracts the contrast information, read the injection details, and the syringe pump details, read the flow rate details and/or reads the pressure data details. Finally, all the information will be written to JSON files by the SR parser module 124.

The combined JSON data created by the axial image parser 120, the OCR module 122 and/or SR parser module 124 is formed into an injector data object 130 and forwarded to a data importer module 132. The data importer module 132 forwards and saves the data into an exam database 134.

In this embodiment, there is also a reference value store 146. Furthermore, there is a reference value interface 148 that can be utilized by a user to visualize and/or edit the reference values stored in the reference value store 146. For constructing a reference value store 146, 15or15 contrast studies distinct value body region or procedure description are being fetched. Then 15or each body region or procedure description there is a provision to set the threshold (reference) value. Via the reference value interface 148 it is possible to add a new procedure description so that threshold can be stored for the same. These threshold values are being stored in the reference value store 146, which is a different cloud store than the exam database 134. The threshold values can be from a single institution and can be region/country specific. Settings of threshold values are different for unit types like contrast volume, flow rate, eGFR etc.

In this embodiment, there is furthermore an alerting rule engine 140. In particular, the alerting rule engine 140 identifies an alert based on the threshold values set for body region, procedure description, eGFR etc. within the reference value store 146. It compares the different values like contrast volume, flow rate of the incoming study (DICOM imaging object 106) processed by the data importer module 132 with that of the threshold value set for the same parameter matching in the study stored in the reference value store 146. Deviation of contrast volume can also be calculated based on body region or procedure description. The same can be indicated in a later visualization of the alert. In particular, the alerting rule engine 140 should be able to identify the alert based on the accumulated or max contrast volume. For a study, if the contrast parameters like volume, flow rate, eGFR exceeds the threshold stored in the reference value store 146 then an alert notification 144 is generated by an alert processing module 142 and notified to the user via mail or any other message. The alert notification 144 can be intimated for a specific alert exam or a set of exams from the scanner. The alert processing module 142 can also store information related to a specific alert in the exam database 134.

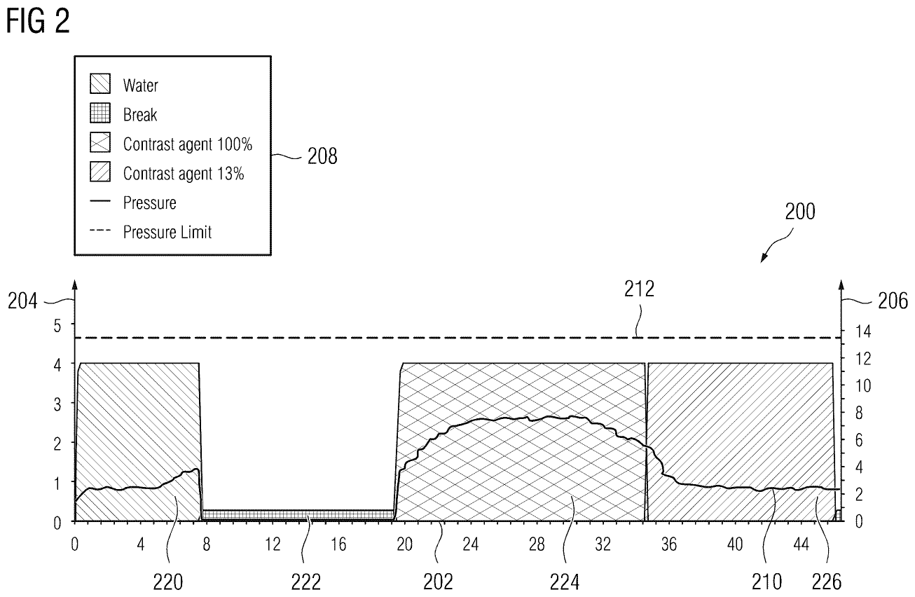

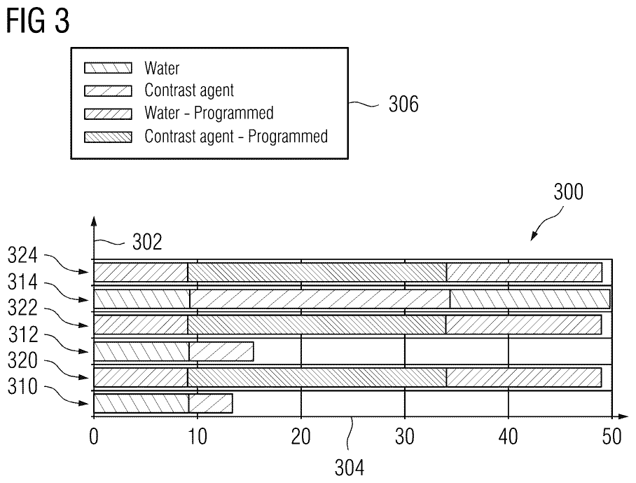

All the data in the exam database 134 can be communicated to a user via an exam data visualization module 136. Alternatively, the data can be transferred via an external system interfacing module 138 to an external database 139, e.g., a HIS (acronym for “Hospital Information System”) or a RIS (acronym for “Radiology Information System”). Details about the visualization will be explained with respect to FIG. 2 and FIG. 3.

Within the embodiment there is also a benchmark calculator 150 that can access the data stored in the exam database 134. In particular, the benchmark calculator 150 can do an analysis of all the data within the exam database 134 to calculate average contrast parameters like volume, flow rate, eGFR per institution or per country/region. These averages or benchmarks can then be stored in a benchmark store 152 and communicated to a user via an benchmark data visualization module 154.

Using a system as depicted in FIG. 1 has the following advantages:

Accuracy and Precision: The system ensures accurate and consistent recording of contrast media details, minimizing the risk of manual errors.

Quality Assurance: The automated capture of contrast media details supports quality assurance initiatives by providing a reliable dataset for analysis and evaluation of imaging protocols and outcomes.

Efficiency: The system streamlines workflow by automating data extraction, saving time for healthcare professionals in the radiology (in particular, CT) department.

Real-time monitoring: The system enables immediate identification and correction of deviations from prescribed contrast administration protocols.

Comprehensive Documentation: The system captures detailed data on contrast volume, flow rates, and injection timing for a more thorough record of each imaging procedure.

Patient Safety and Compliance: The system enhances patient safety, supports regulatory compliance, and facilitates quality assurance through standardized and automated data capture.

Comparison/Benchmarking: With the capability of the system to handle multi-tenancy, the analytics across institutions could be provided hence supporting institutions to improve.

FIG. 2 displays a first visualization 200 of the contrast agent admitted within one medical imaging procedure. In particular, the first visualization 200 can be created via the exam data visualization module 136 based on the data within the exam database 134. In particular, the first visualization is based on a structured report in the exam database 134, and is the graphical visualization of flow rate and pressure data of the contrast media 224, 226, saline/water 220 and the break/hold duration 222 during the injection sequence across its entirety.

The flowrate data of the injection sequence is represented using the line chart format with the area style coloured based on the sequence action at that instance. The time period is represented in seconds along the X-axis 202 and the flow rate 220, 224, 226 is represented in mi/s along the Y-axis 204.

The pressure data of the injection sequence is represented using the line chart format. The time period is represented in seconds along the X-axis 202 and the pressure 210 is represented along the Y-axis 206. One can also represent the pressure 212 limit data as a constant value until one can derive this data from the JSON.

The flow rate 220, 224, 226 and pressure data 210, 212 are overlapped on the same chart for easier understanding of the sequence. The Y-axis 204 on the left side of the chart represents the range of the flow rate data while the Y-axis 206 on the right side represents the range of the pressure data. contrast media 224, 226 consumed is represented using a shade of green. In a sequence, there can be multiple contrast media consumptions based on factors like concentration. Data for each of these consumptions are distinctively represented. For example, in this embodiment the first contrast media curve 224 corresponds to a concentration of 100% and the second contrast medica curve 226 corresponds to a concentration of 13% as indicated in the legend 208. Saline or water 220 is represented using a shade of blue. Break or hold phase 222 is represented using a shade of orange. Pressure data 210, 212 is visualized as a line using a shade of red. The display of each of these can be toggled using the legend 208 that is provided in the chart.

FIG. 3 displays a second visualization 300 of the contrast agent admitted within one medical imaging procedure. In particular, the second visualization 300 can be created via the exam data visualization module 136 based on the data within the exam database 134. In particular, the first visualization is based on a structured report in the exam database 134, and is the graphical visualization of the volume consumed and injection period of the contrast media, saline/water and the break/hold duration during the injection sequence across its entirety.

The representation provides data of the performed execution 310, 312, 314 and the planned execution 320, 322, 324 of the sequence. The chart takes only the duration of the sequence for consideration with the time period represented along the X-axis 304. The Y-axis 302 represents the sequence itself with the performed sequences 310, 312, 314 and planned sequence 320, 322, 324 grouped together for easier understanding.

The performed sequence 310, 312, 314 is labelled as an “Actual” sequence and planned sequence 320, 322, 324 is labelled as a “Programmed” sequence with the numeric sequence identifier next to them. The planned sequence 320, 322, 324 is represented in a darker shade compared to the performed sequence 310, 312, 314. The duration and the volume applied of each sequence is represented for the all the factors involved in the sequence. A legend 306 is provided to convey the respective information to the user of the second visualization 300.

Contrast media consumed is represented using a shade of green. Saline or water is represented using a shade of blue. Break or hold phase is represented using a shade of orange. There can be a representation for varying contrast concentration which can be used for further analysis.

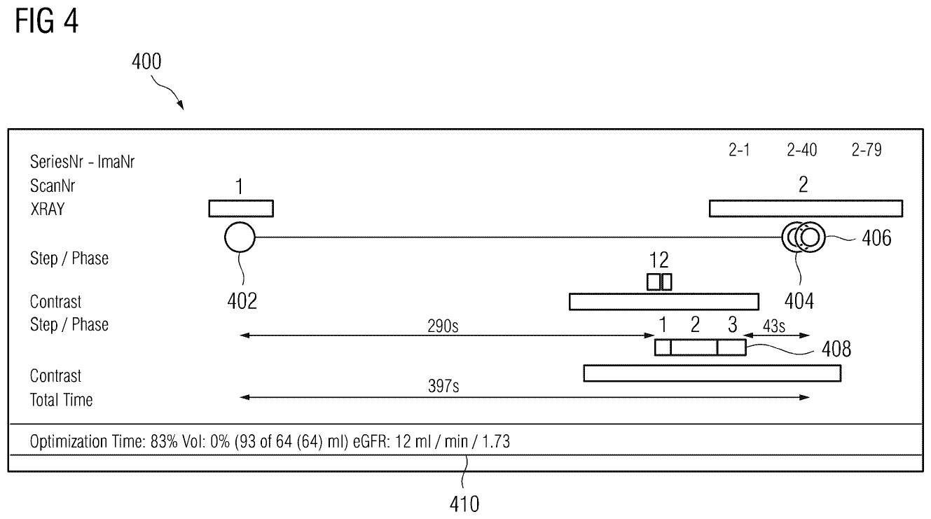

FIG. 4 displays a third visualization 400 of the contrast agent admitted within one medical imaging procedure. In particular, the third visualization 400 can be created via the exam data visualization module 136 based on the data within the exam database 134. The third visualization 400 displays a combination of the scanner events 402, 404, 406 (i.e., irradiation events) along with the contrast injection sequence representation 408. Contrast flows are synced with the scans i.e., each event represents the image capture event. White circle 402, 408 expresses the start and end point of the scan. Red circle 406 expresses major scan. Dots within the circles represent the image capture event. First row of the third visualization indicates the series and image number, below which the scan number can be indicated. Based on the third visualization 400, optimization of time and contrast volume saving can be done. In particular, volume optimization can be obtained by measuring the contrast media consumption post the last scan before the flush. This contrast media consumed can be saved and hence optimization of the contrast agent volume consumed can be achieved.

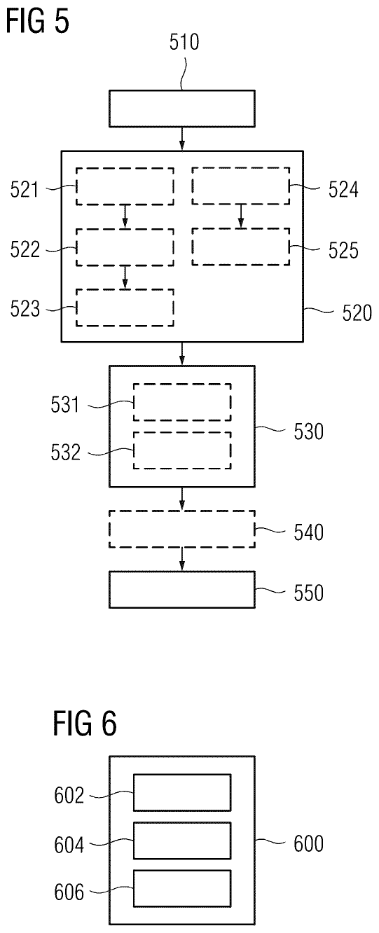

FIG. 5 displays a flowchart of an embodiment of a method for providing a contrast agent alert 144. The method comprises the step of receiving 510 medical imaging data 106 related to a medical imaging procedure, wherein during the medical imaging procedure contrast agent was administered to a patient. The method furthermore comprises determining 520 a volume of contrast agent administered to the patient based on the medical imaging data 106. The method furthermore comprises determining 530 a reference volume of contrast agent based on the medical imaging data 106. The method furthermore comprises providing 550 an alert 144 if the volume of contrast agent administered fulfills a condition, wherein the condition is based on the reference volume of contrast agent.

In a first variant of this embodiment, the step of determining 520 the volume of contrast agent administered to the patient comprises the step of performing 521 an optical character recognition within the secondary capture image 112 and the step of determining 522 the volume of contrast agent administered based on recognized characters within the secondary capture image. In particular, this variant is relevant of the medical imaging data 106 comprises a secondary capture image 112. Advantageously, the determining 520 the volume of contrast agent administered to the patient furthermore comprises the step of determining 523 a planned volume of contrast agent based on recognized characters within the secondary capture image 112. Herein the condition and/or the reference volume of contrast agent determined in step 530 or 550 is based on the planned volume of contrast agent.

In a second variant of this embodiment, the step of determining 520 the volume of contrast agent administered to the patient comprises determining 525 at least one of the volume of contrast agent administered and the reference volume of contrast agent based on an axial image 110, In case the medical imaging data 106 comprises at least two axial images 110, the step of determining 520 the volume of contrast agent administered to the patient comprises—selecting 524 one of the at least two axial images 110 based on a contrast bolus time and determining 525 at least one of the volume of contrast agent administered and the reference volume of contrast agent based on the selected one of the at least two axial images 110.

In particular, the additional steps of the first variant are executed of there is a secondary capture image 112 within the medical imaging data 106, and the additional steps of the second variant are executed if there is at least one axial image 110 within the medical imaging data 106. In cases where the medical imaging data 106 comprises both a secondary capture image 112 and at least one axial image 110 the additional steps of both the first and the second variant can be executed.

Advantageously the step of determining 530 a reference volume of contrast agent comprises the step of determining 531 imaging metadata based on the based on the medical imaging data 106 and querying 532 a database 146 (in particular, the reference value store 146) comprising a plurality of reference volumes of contrast agent based on the imaging metadata. In particular, if the medical imaging data 106 comprises DICOM images, the image metadata can comprise metatags of the DICOM images.

FIG. 6 displays a system 600 for providing a contrast agent alert 144 according to an embodiment of the invention. The system 600 is configured for executing a method for providing a contrast agent alert 144 according to embodiments of the invention.

The system 600 can be or comprise a (personal) computer, a workstation, a virtual machine running on host hardware, a microcontroller, or an integrated circuit. As an alternative, the system 600 can be a real or a virtual group of computers (the technical term for a real group of computers is “cluster”, the technical term for a virtual group of computers is “cloud”).

The system 600 can comprise an interface 602, a computation unit 604 and a memory unit 606. An interface 602 can be a hardware interface or as a software interface (e.g., PCIBus, USB or Firewire). A computation unit 604 can comprise hardware elements and software elements, for example a microprocessor, a CPU (acronym for “central processing unit”), a GPU (acronym for “graphical processing unit”), a field programmable gate array (an acronym is “FPGA”) or an ASIC (acronym for “application-specific integrated circuit”). A computation unit 604 can be configured for multithreading, i.e., the computation unit can host different computation processes at the same time, executing the either in parallel or switching between active and passive computation processes. In particular, the computation unit 604 can be denoted as processor. The memory unit 606 can comprise one or more databases. Each of the interface 602, the computation unit 604 and the memory unit 606 can comprise several subunits which are configured to executed different tasks and/or which are spatially separated.

The system 600 can be connected to one or more databases (in particular, the exam database 134, the reference value store 146 and/or benchmark store 152) via a network. The network can be realized as a LAN (acronym for “local area network”), in particular a WiFi network, or any other local connection. Alternatively, the network can be the internet. In particular, the network could be realized as a VPN (acronym for “virtual private network”). Alternatively, the database can also be integrated into the system 600, e.g., the database could be stored within the memory unit 606 of the providing system 600. In this case the database is connected with an internal connection.

Wherever not already described explicitly, individual embodiments, or their individual aspects and features, can be combined or exchanged with one another without limiting or widening the scope of the described invention, whenever such a combination or exchange is meaningful and in the sense of this invention. Advantages which are described with respect to one embodiment of the present invention are, wherever applicable, also advantageous of other embodiments of the present invention.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A computer-implemented method for providing a contrast agent alert, comprising:
receiving medical imaging data related to a medical imaging procedure, wherein a contrast agent was administered to a patient during the medical imaging procedure;
determining a volume of contrast agent administered to the patient based on the medical imaging data;
determining a reference volume of contrast agent based on the medical imaging data; and
providing an alert if the volume of contrast agent administered fulfills a condition, wherein the condition is based on the reference volume of contrast agent.

2. The method of claim 1, wherein the medical imaging data comprises a secondary capture image, wherein the determining the volume of contrast agent administered comprises:
performing an optical character recognition within the secondary capture image, and
determining the volume of contrast agent administered based on recognized characters within the secondary capture image.

3. The method of claim 2, further comprising:
determining a planned volume of contrast agent based on recognized characters within the secondary capture image, wherein at least one of the condition or the reference volume of contrast agent is based on the planned volume of contrast agent.

4. The method of claim 1, wherein the medical imaging data comprises a structured report document and the structured report document comprises a first data element related to the volume of contrast agent administered.

5. The method of claim 4, wherein the structured report document comprises a second data element related to a planned volume of contrast agent, wherein at least one of the condition or the reference volume of contrast agent is based on the planned volume of contrast agent.

6. The method of claim 4, wherein
the structured report document further comprises a second data element related to at least one of injection details, syringe pump details, flow rate details, or pressure data details, and
at least one of the volume of contrast agent administered or the reference volume of contrast agent is determined based on the second data element.

7. The method of claim 1, wherein the medical imaging data comprises an axial image, the method further comprising:
determining at least one of the volume of contrast agent administered or the reference volume of contrast agent based on the axial image.

8. The method of claim 1, wherein the medical imaging data comprises at least two axial images, the method further comprising:
selecting one of the at least two axial images based on a contrast bolus time; and
determining at least one of the volume of contrast agent administered and the reference volume of contrast agent based on the selected one of the at least two axial images.

9. The method of claim 1, wherein the determining the reference volume of contrast agent comprises:
determining imaging metadata based on the medical imaging data, and
querying a database comprising a plurality of reference volumes of contrast agent based on the imaging metadata.

10. The method of claim 9, wherein the imaging metadata corresponds to a procedure description of the medical imaging procedure.

11. The method of claim 1, furthermore comprising:
determining a body region being subject of the medical imaging procedure, wherein the condition is furthermore based on the body region.

12. The method of claim 1, wherein the condition is based on at least one of an accumulated volume of contrast agent administered or maximal volume of contrast agent administered.

13. The method of claim 1, furthermore comprising:
anonymizing or pseudonymizing the medical imaging data.

14. A system for providing a contrast agent alert, comprising:
at least one processor configured to cause the system to,
receiving medical imaging data related to a medical imaging procedure, wherein a contrast agent was administered to a patient during the medical imaging procedure,
determining a volume of contrast agent administered to the patient based on the medical imaging data,
determining a reference volume of contrast agent based on the medical imaging data, and
providing an alert if the volume of contrast agent administered fulfills a condition, wherein the condition is based on the reference volume of contrast agent.

15. A non-transitory computer-readable storage medium comprising instructions which, when executed by a system, cause the system to perform the method of claim 1.

16. A non-transitory computer-readable storage medium comprising instructions which, when executed by a system, cause the system to perform the method of claim 2.

17. The method of claim 5, wherein
the structured report document further comprises a second data element related to at least one of injection details, syringe pump details, flow rate details, or pressure data details, and
at least one of the volume of contrast agent administered or the reference volume of contrast agent is determined based on the second data element.

18. The method of claim 2, wherein the medical imaging data comprises an axial image, the method further comprising:
determining at least one of the volume of contrast agent administered or the reference volume of contrast agent based on the axial image.

19. The method of claim 2, wherein the medical imaging data comprises at least two axial images, the method further comprising:
selecting one of the at least two axial images based on a contrast bolus time; and
determining at least one of the volume of contrast agent administered and the reference volume of contrast agent based on the selected one of the at least two axial images.

20. The method of claim 2, wherein the determining the reference volume of contrast agent comprises:
determining imaging metadata based on the medical imaging data, and querying a database comprising a plurality of reference volumes of contrast agent based on the imaging metadata.
